(19) United States
(12) Patent Application Publication
(10) Pub. No.: US 2020/0163728 A1
(43) Pub. Date: May 28, 2020

(54) STERILE BARRIER SYSTEM FOR ARTICULATING ROBOTIC INTRODUCER SYSTEM ASSEMBLY

(71) Applicant: MEDROBOTICS CORPORATION, Rayham, MA (US)

(72) Inventors: William A. Dalpe, Rehoboth, MA (US); David Zitnick, Providence, RI (US); Andras Pungor, Bridgewater, MA (US); Leland Witherspoon, Elk Grove, CA (US)

(21) Appl. No.: 16/611,617

(22) PCT Filed: May 9, 2018
(86) PCT No.: PCT/US18/31774

§ 371 Date: Nov. 7, 2019

Related U.S. Application Data
(60) Provisional application No. 62/504,175, filed on May 10, 2017.

(51) Int. Cl. A61B 34/30; A61B 46/10

(52) U.S. Cl. CPC A61B 34/30; A61B 90/50; A61B 46/10

(57) ABSTRACT

A system for performing a medical procedure comprises a robotic introducer assembly including: a base unit, the base unit including a proximal base portion and a base extension extending from the proximal base portion; and a probe assembly operably and removably coupled to the base unit. The probe assembly includes an articulating probe. The base unit is constructed and arranged to control a movement of the articulating probe. The system further comprises a sterile barrier system positioned over at least a portion of the robotic introducer assembly, the sterile barrier system providing isolation between any non-sterile portion of the robotic introducer assembly and the sterile field or any sterile operators (e.g. to prevent exposure of any non-sterile portion to the sterile field and/or sterile operators). The sterile barrier system comprises a sterile frame having a first end coupled to a distal portion of the robotic introducer assembly and a second end coupled to a portion of the robotic introducer assembly proximal to a distal portion of the base unit. A sterile drape extends from the sterile frame and constructed and arranged for positioning about the base unit.

### RELATED APPLICATIONS

This application claims benefit of U.S. Provisional Application No. 62/504,175, filed May 10, 2017, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, the content of which is incorporated herein by reference in its entirety.

This application is related to U.S. patent application Ser. No. 14/892,750, filed Nov. 20, 2015, the content of which is incorporated herein by reference in its entirety.

This application is related to U.S. patent application Ser. No. 15/899,826, filed Feb. 20, 2018, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, the content of which is incorporated herein by reference in its entirety.

This application is related to U.S. patent application Ser. No. 13/880,525, filed Apr. 19, 2013, now U.S. Pat. No. 8,992,421, the content of which is incorporated herein by reference in its entirety.

This application is related to U.S. patent application Ser. No. 14/587,166, filed Dec. 31, 2014, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US12/40414, filed Jun. 1, 2012, the content of which is incorporated herein by reference in its entirety.

This application is related to U.S. patent application Ser. No. 14/119,316, filed Nov. 21, 2013, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, the content of which is incorporated herein by reference in its entirety.

This application is related to U.S. patent application Ser. No. 13/884,407, filed May 9, 2013, now U.S. Pat. No. 9,649,163, the content of which is incorporated herein by reference in its entirety.

This application is related to U.S. patent application Ser. No. 15/587,832, filed May 5, 2017, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US 12/32279, filed Apr. 5, 2012, the content of which is incorporated herein by reference in its entirety.

This application is related to U.S. patent application Ser. No. 14/008,775, filed Sep. 30, 2013, now U.S. Pat. No. 9,962,179, the content of which is incorporated herein by reference in its entirety.

This application is related to U.S. patent application Ser. No. 14/944,665, filed Nov. 18, 2015, the content of which is incorporated herein by reference in its entirety.

This application is related to U.S. patent application Ser. No. 14/945,685, filed Nov. 19, 2015, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US 12/54802, filed Sep. 12, 2012, the content of which is incorporated herein by reference in its entirety.

This application is related to U.S. patent application Ser. No. 14/343,915, filed Mar. 10, 2014, now U.S. Pat. No. 9,757,856, issued Sep. 12, 2017, the content of which is incorporated herein by reference in its entirety.

This application is related to U.S. patent application Ser. No. 15/064,043, filed Mar. 8, 2016, now U.S. Pat. No. 9,572,628, issued Feb. 21, 2017, the content of which is incorporated herein by reference in its entirety.

This application is related to U.S. patent application Ser. No. 15/684,268, filed Aug. 23, 2017, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, the content of which is incorporated herein by reference in its entirety.

This application is related to U.S. patent application Ser. No. 13/812,324, filed Jan. 25, 2013, now U.S. Pat. No. 9,901,410, issued Feb. 27, 2018, the content of which is incorporated herein by reference in its entirety.

This application is related to U.S. patent application Ser. No. 15/874,189, filed Jan. 18, 2018, the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US 12/70924, filed Dec. 20, 2012, the content of which is incorporated herein by reference in its entirety.

This application is related to U.S. patent application Ser. No. 14/364,195, filed Jun. 10, 2014, now U.S. Pat. No. 9,364,955 issued Jun. 14, 2016, the content of which is incorporated herein by reference in its entirety.

This application is related to U.S. patent application Ser. No. 15/180,503, filed Jun. 13, 2016, now U.S. Pat. No. 9,821,477, issued Nov. 21, 2017, the content of which is incorporated herein by reference in its entirety.

This application is related to U.S. patent application Ser. No. 15/786,901, filed Oct. 18, 2017, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US 13/54326, filed Aug. 9, 2013, the content of which is incorporated herein by reference in its entirety.

This application is related to U.S. patent application Ser. No. 14/418,993, filed Feb. 2, 2015, now U.S. Pat. No. 9,675,380 issued Jun. 13, 2017, the content of which is incorporated herein by reference in its entirety.

This application is related to U.S. patent application Ser. No. 15/619,875, filed Jun. 12, 2017, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US14/10808, filed Jan. 9, 2014, the content of which is incorporated herein by reference in its entirety.

This application is related to U.S. patent application Ser. No. 14/759,020, filed Jan. 9, 2014, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US13/43858, filed Jun. 3, 2013, the content of which is incorporated herein by reference in its entirety.

This application is related to U.S. patent application Ser. No. 14/402,224, filed Nov. 19, 2014, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US13/38701, filed May 20, 2014, the content of which is incorporated herein by reference in its entirety.

This application is related to U.S. patent application Ser. No. 14/888,541, filed Nov. 2, 2015, now U.S. Pat. No. 9,517,059, issued Dec. 13, 2016, the content of which is incorporated herein by reference in its entirety.

This application is related to U.S. patent application Ser. No. 15/350,549, filed Nov. 14, 2016, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US14/36571, filed May 2, 2014, the content of which is incorporated herein by reference in its entirety.

This application is related to U.S. patent application Ser. No. 14/888,189, filed Oct. 30, 2015, now U.S. Pat. No. 9,913,695, issued Mar. 13, 2018, the content of which is incorporated herein by reference in its entirety.

This application is related to U.S. patent application Ser. No. 14/916,664, filed Mar. 9, 2018, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US 14/67091, filed Nov. 24, 2014, the content of which is incorporated herein by reference in its entirety.

This application is related to U.S. patent application Ser. No. 15/038,531, filed May 23, 2016, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US15/34424, filed Jun. 5, 2015, the content of which is incorporated herein by reference in its entirety.

This application is related to U.S. patent application Ser. No. 15/315,868, filed Dec. 2, 2016, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US16/28374, filed Apr. 20, 2016, the content of which is incorporated herein by reference in its entirety.

This application is related to U.S. patent application Ser. No. 15/567,109, filed Oct. 17, 2017, the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US17/54297, filed Sep. 29, 2017, the content of which is incorporated herein by reference in its entirety.

This application is related to U.S. Provisional Application No. 62/517,433, filed Jun. 9, 2017, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US Ser. No. 18/026,016, filed Apr. 4, 2018, the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to U.S. Provisional Application No. 62/613,899, filed Jan. 5, 2018, the content of which is incorporated herein by reference in its entirety.

This application is related to U.S. Provisional Application No. 62/614,223, filed Jan. 5, 2018, the content of which is incorporated herein by reference in its entirety.

This application is related to U.S. Design Application No. 29/632,148, filed Jan. 5, 2018, the content of which is incorporated herein by reference in its entirety.

This application is related to U.S. Provisional Application No. 62/614,224, filed Jan. 5, 2018, the content of which is incorporated herein by reference in its entirety.

This application is related to U.S. Provisional Application No. 62/614,228, filed Jan. 5, 2018, the content of which is incorporated herein by reference in its entirety.

This application is related to U.S. Provisional Application No. 62/614,225, filed Jan. 5, 2018, the content of which is incorporated herein by reference in its entirety.

This application is related to U.S. Provisional Application No. 62/614,240, filed Jan. 5, 2018, the content of which is incorporated herein by reference in its entirety.

This application is related to U.S. Provisional Application No. 62/614,235, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### TECHNICAL FIELD

The present inventive concepts relate generally to the field of surgical instruments, and more particularly, to sterile barrier systems for a medical device having an articulating robotic arm for performing medical procedures.

### BACKGROUND

As less invasive medical techniques and procedures become more widespread, medical professionals such as surgeons may require articulating probes and other surgical tools, such as robotically controlled probes, to perform such less invasive medical techniques and procedures that access interior regions of the body via a body orifice such as the mouth. It is desirable for some medical devices, such as expensive robotic surgical instruments to be reusable. However, cleaning and resterilization of some surgical instruments may be difficult, such as those instruments having both reusable and disposable components.

### SUMMARY

In one aspect, provided is a system for performing a medical procedure comprising a robotic introducer assembly including: a base unit, the base unit including a proximal base portion and a base extension extending from the proximal base portion; and a probe assembly operably and removably coupled to the base unit. The probe assembly includes an articulating probe. The base unit is constructed and arranged to control a movement of the articulating probe. The system further comprises a sterile barrier system positioned over at least a portion of the robotic introducer assembly, the sterile barrier system providing isolation between any non-sterile portion of the robotic introducer assembly and the sterile field or any sterile operators (e.g. to prevent exposure of any non-sterile portion to the sterile field and/or sterile operators). The sterile barrier system comprises a sterile frame having a first end coupled to a distal portion of the robotic introducer assembly and a second end coupled to a portion of the robotic introducer assembly proximal to a distal portion of the base unit. A sterile drape extends from the sterile frame and constructed and arranged for positioning about the base unit.

In an embodiment, the articulating probe translates relative to the sterile frame.

In an embodiment, the articulating probe translates through the sterile drape.

In an embodiment, the articulating probe translates relative to the sterile frame so that the probe extends through the frame and a portion of the probe assembly is covered by the sterile drape and a portion of the probe assembly extends through an interior region of the frame.

In an embodiment, the sterile frame includes an opening constructed and arranged to expose at least a portion of the probe assembly.

In an embodiment, the sterile frame includes a relief feature for allowing the probe assembly to extend through the relief feature for uncoupling from the base unit.

In an embodiment, the sterile drape includes a perforation that extends in a same longitudinal direction as the relief feature of the sterile frame, wherein the probe assembly is removed through an opening and the relief feature in the sterile frame and the perforation is separated.

In an embodiment, the relief feature includes at least one of an opening, a hinge, a different material, a thinner region or a weaker region than a peripheral body of the frame.

In an embodiment, the frame includes an interior region including a pass-through region wide enough for receiving a portion of the probe assembly.

In an embodiment, the sterile drape includes an opening, and wherein the probe assembly passes through an opening in the frame.

In an embodiment, the sterile barrier system creates a barrier between sterile and non-sterile components of the robotic introducer assembly.

In an embodiment, the sterile barrier system includes a top portion of the probe assembly.

In an embodiment, the probe assembly and sterile barrier system are each constructed and arranged for use in fewer medical procedures than the base unit.

In an embodiment, the probe assembly and sterile barrier system are each sterile at a time of coupling to the base unit, and the base unit is non-sterile.

In an embodiment, the base unit is reusable and the probe assembly is disposable.

In an embodiment, each of the frame and drape of the sterile barrier system is sterile and single use.

In an embodiment, the sterile barrier system further includes a lower portion operably attached to frame via a hinge.

In an embodiment, the hinge is configured to rotatably attach the lower portion to the frame.

In an embodiment, the sterile frame engages at least one of a mid-portion of the probe assembly or a distal end of the proximal base portion, and wherein the lower portion couples to the frame to collectively surround the base extension and a portion of the probe assembly on the base extension.

In an embodiment, the probe assembly further comprises: a housing surrounding at least a portion of the articulating probe, the articulating probe configured to movably extend from the housing and the base extension.

In an embodiment, the base unit includes a drive assembly including a plurality of drive capstans that mate with corresponding cable bobbins of a pulley assembly of the articulating probe.

In an embodiment, the drive capstans of the base unit drive steering cables that are steerable by a cable control assembly, which advance or retract inner or outer links with respect to one another during manipulation of the probe assembly.

In an embodiment, the steering cables can be used to releasably tighten to lock or stiffen either or both of the plurality of inner links or the plurality of outer links.

In an embodiment, the base extension includes a plurality of fingers for translating the articulating probe.

In an embodiment, the system further comprises a camera assembly.

In an embodiment, the camera assembly is at least one of disposable or reusable, but used in fewer medical procedures than the base unit.

In an embodiment, the camera assembly includes a camera, a camera connector, and a camera cable extending between the camera and the camera connector.

In an embodiment, the base unit includes a connection port that receives the camera connector, and wherein the camera assembly is external to the sterile barrier system.

In an embodiment, the camera connector is connected to the connection port after the probe assembly is coupled to the base unit.

In an embodiment, the system further comprises a carrier for the articulating probe, the carrier comprising packaging for a shipment of the disposable probe assembly.

In an embodiment, a proximal portion of the probe assembly includes one or more bobbins, and wherein the carrier packaging includes an insert that prevents the bobbins from rotation during the shipment.

In an embodiment, the sterile drape is constructed and arranged for positioning about the proximal base portion and the base extension of the base unit.

In an embodiment, the sterile drape is coupled to a perimeter of the sterile frame.

In an embodiment, the sterile drape covers the proximal base portion of the base unit and at least a portion of the base extension of the base unit.

In an embodiment, the second end of the sterile frame is coupled to at least one of a portion of the base extension or a mid-portion of the probe assembly.

In an embodiment, the sterile frame includes at the first end: a notch that mates with a guide pin extending from the base extension; and a clip at a retention portion of the frame for coupling to the base extension.

In an embodiment, the second end of the sterile frame includes one or more projections at engagement portions of the frame for coupling to the base unit.

In an embodiment, the first end of the sterile frame is coupled to a distal end of the base extension.

In an embodiment, the sterile frame includes a covering formed of an elastomeric material positioned about some or all of the opening of the sterile frame between the first end and the second end of the frame that conforms to at least a portion of the probe assembly.

In an embodiment, the covering includes a region that conforms to one or more quick release buttons extending from the base unit.

In an embodiment, the one or more quick release buttons disconnect the probe from the base unit when depressing the elastomeric material over the one or more quick release buttons.

In an embodiment, the covering includes a grommet that forms a sterile seal about a camera assembly extending from the base unit.

In another aspect, a method of assembling a robotic introducer system, comprises coupling a probe assembly to a base unit of a robotic introducer assembly; coupling a camera assembly to the base unit; inserting the probe assembly through an opening in a sterile frame of a barrier system; coupling a first end of the sterile frame about the probe assembly to a distal end of the robotic introducer assembly; coupling a second end of the sterile frame to a portion of the robotic introducer assembly proximal to a distal portion of the base unit; completing the assembly of the robotic introducer system by positioning a sterile drape of the barrier system extending from the sterile frame about the base unit of the robotic introducer assembly.

In another aspect, a system for performing a medical procedure comprises a robotic introducer assembly including: a base unit, the base unit including: a proximal base portion; and a base extension extending from the proximal base portion; and a probe assembly operably and removably coupled to the base unit, the probe assembly including an articulating probe, wherein the base unit is constructed and arranged to control a movement of the articulating probe. The system further comprises a sterile barrier system positioned over at least a portion of the robotic introducer assembly, the sterile barrier system providing isolation between any non-sterile portion of the robotic introducer assembly and the sterile field or any sterile operators (e.g. to prevent exposure of any non-sterile portion to the sterile field and/or sterile operators). The sterile barrier system comprises a sterile frame assembly comprising a first sterile frame and a second sterile frame positioned about a distal portion of the robotic introducer assembly; and a sterile drape coupled to a third sterile frame extending from the sterile frame and constructed and arranged for interfacing with the first and second sterile frames at a portion of the robotic introducer assembly proximal to a distal portion of the base unit.

In an embodiment, the second sterile frame is configured to removably attach to the underside of the base extension and the first sterile frame is configured to cover a top region of the base extension where a linear motion transfer is made from the base unit to the probe assembly along the base extension.

In an embodiment, the articulating probe translates relative to the sterile frame assembly.

In another aspect, a method of assembling a robotic introducer system comprises coupling a probe assembly to a base unit of a robotic introducer system; axially coupling a bottom sterile frame to a base extension of a robotic introducer assembly; coupling a top sterile frame to the bottom sterile frame, the top and bottom sterile frame surrounding the base extension; positioning a probe assembly of the robotic introducer assembly through an opening between the top and bottom sterile frames; positioning a third sterile frame about the probe assembly and the base extension for interfacing with the first and second sterile frames at a portion of the robotic introducer assembly proximal to a distal portion of the base unit; and positioning a sterile drape of the barrier system extending from the third sterile frame about the base unit.

In an embodiment, the method further comprises removing the probe assembly from the base unit including: separating the top sterile frame from the bottom sterile frame; and removing the probe assembly through an opening in the third sterile frame, the top sterile frame removed.

In an embodiment, the method further comprises coupling a camera assembly to the base unit after coupling the bottom sterile frame to the base extension and before coupling the top sterile frame to the bottom sterile frame.

In another aspect, a system for performing a medical procedure comprises a robotic introducer assembly including: a base unit, the base unit including: a proximal base portion; and a base extension extending from the proximal base portion; and a probe assembly operably and removably coupled to the base unit, the probe assembly including an articulating probe, wherein the base unit is constructed and arranged to control a movement of the articulating probe; and the system further comprising a sterile barrier system positioned over at least a portion of the robotic introducer assembly, providing isolation between any non-sterile portion of the robotic introducer assembly and the sterile field or any sterile operators (e.g. to prevent exposure of any non-sterile portion to the sterile field and/or sterile operators). The sterile barrier system comprising: a first portion comprising a first sterile drape coupled to a first frame, the first portion constructed and arranged to surround the base extension; and a second portion comprising a second sterile drape coupled to a second sterile frame constructed and arranged to interface with the first sterile frame, the second sterile drape constructed and arranged to surround the proximal base portion.

In an embodiment, the first frame is a non-sterile frame that covers a top portion of the base extension, and wherein the first sterile drape covers a bottom portion of the base extension.

In an embodiment, the articulating probe translates relative to the first frame.

In an embodiment, the first sterile frame comprises a projection configured to interface with the second sterile frame to form a seal between the first and second portions of the sterile barrier system.

In another aspect, a method of assembling a robotic introducer system comprises coupling a probe assembly to a base unit of a robotic introducer assembly; coupling a camera assembly to the base unit; coupling a first portion of a sterile barrier system to a base extension of the base unit of the robotic introducer assembly, the first portion including a first frame and a first sterile drape that surrounds the base extension; and coupling a second portion of the sterile barrier system to the first portion, the second portion including a second frame and a second sterile drape, the second sterile drape surrounding a proximal base portion of the base unit.

In another aspect, a system for performing a medical procedure comprises a robotic introducer assembly including: a base unit, the base unit including: a proximal base portion; and a base extension extending from the proximal base portion; and a probe assembly operably and removably coupled to the base unit, the probe assembly including an articulating probe, wherein the base unit is constructed and arranged to control a movement of the articulating probe; and the system further comprising: a first sterile drape positioned over a top portion of the base extension; and a second sterile drape comprising a bag like structure constructed and arranged to surround the proximal base portion, the second sterile drape having a proximal end opening and a distal end opening, and an elastic cinch surrounding the distal end opening, the elastic cinch elastically engaging the base extension.

In an embodiment, the method comprises the robotic introducer system including additional features as claimed.

In another aspect, provided is a robotic introducer system as described in reference to the figures.

In another aspect, provided is a method of using a robotics introducer system as described in reference to the figures.

In another aspect, provided is a method of performing a medical procedure as described in reference to the figures.

### BRIEF DESCRIPTION OF THE DRAWINGS

Advantages of examples of the present inventive concepts may be better understood by referring to the following description in conjunction with the accompanying drawings, in which like numerals indicate like structural elements and features in various figures. The drawings are not necessarily to scale; emphasis instead being placed upon illustrating the principles of features and implementations.

FIG. 1 is a perspective view of a robotic introducer system comprising a disposable probe assembly and a sterile barrier system separate from a base unit, in accordance with embodiments of the present inventive concepts.

FIG. 1A is a perspective view of a robotic introducer assembly, in accordance with embodiments of the present inventive concepts.

FIG. 1B is a perspective view of the disposable probe assembly and partially transparent view of the sterile barrier system coupled to the robotic introducer assembly of FIG. 1A, in accordance with embodiments of the present inventive concepts.

FIG. 2 is a perspective view of a sterile frame of the sterile barrier system of FIGS. 1 and 1B, in accordance with embodiments of the present inventive concepts.

FIG. 3 is a perspective view of quick release buttons of the base unit of FIG. 1, in accordance with embodiments of the present inventive concepts.

FIG. 4A is a perspective view of the front portion of the sterile frame of the sterile barrier system of FIGS. 1-3, in accordance with the embodiments of the present inventive concepts.

FIG. 4B is a perspective view of the front portion of the sterile frame of the sterile barrier system of FIGS. 1-4A, interlocked with the disposable probe and base unit, in accordance with embodiments of the present inventive concepts.

FIG. 5 is a flowchart of a method for assembling a robotic introducer assembly of FIGS. 1A and 1B, in accordance with the embodiments of the present inventive concepts.

FIG. 6 is a perspective view of a disposable assembly and a sterile barrier system separate from a base unit, in accordance with embodiments of the present inventive concepts.

FIG. 7A is a perspective view of an assembled robotic introducer assembly, comprising a disposable probe assembly operably attached to a base unit, in accordance with embodiments of the present inventive concepts.

FIG. 7B is a perspective view of a robotic introducer assembly with a first portion of a sterile barrier system, shown partially transparent, attached to a portion of a robotic introducer system, in accordance with embodiments of the present inventive concepts.

FIG. 7C is a perspective view of a robotic introducer assembly with a first portion of a sterile barrier system further secured to an introducer assembly, in accordance with embodiments of the present inventive concepts.

FIG. 7D is a perspective view of a robotic introducer assembly with a second portion of a sterile barrier system further secured to an introducer assembly, in accordance with embodiments of the present inventive concepts.

FIG. 8 is a flowchart of a method for assembling a robotic introducer assembly, in accordance with embodiments of the present inventive concepts.

FIG. 9 is a perspective view of a disposable probe assembly and a sterile barrier system separate from a base unit, in accordance with embodiments of the present inventive concepts.

FIG. 10A is perspective view of a lower frame of a sterile barrier system coupled to a base unit, in accordance with embodiments of the present inventive concepts.

FIG. 10B is a photograph of a lower frame coupled to a base unit, in accordance with embodiments of the present inventive concepts.

FIG. 10C is a perspective view of an assembled robotic introducer assembly, comprising a disposable probe assembly operably attached to a base unit, in accordance with embodiments of the present inventive concepts.

FIG. 10D is a perspective view of an upper frame of a sterile barrier system coupled to an assembled robotic introducer assembly, in accordance with embodiments of the present inventive concepts.

FIG. 10E is a perspective view of a drape of a sterile barrier system coupled to an assembled robotic introducer assembly, in accordance with embodiments of the present inventive concepts.

FIG. 11 is a flowchart of a method for assembling a robotic introducer assembly, in accordance with embodiments of the present inventive concepts.

FIG. 12 is a perspective view of a disposable probe assembly and a sterile barrier system separate from a base unit, in accordance with embodiments of the present inventive concepts.

FIG. 13A is a perspective view of a robotic introducer assembly with a first portion of a sterile barrier system, shown partially transparent, attached to a portion of a base unit, in accordance with embodiments of the present inventive concepts.

FIG. 13B is a perspective view of an assembled robotic introducer assembly, comprising a disposable probe assembly operably attached to a base unit, in accordance with embodiments of the present inventive concepts.

FIG. 13C is a perspective view of a robotic introducer assembly with a second portion of a sterile barrier system further secured to a robotic introducer assembly, in accordance with embodiments of the present inventive concepts.

FIG. 14 is a flowchart of a method for assembling a robotic introducer assembly, in accordance with embodiments of the present inventive concepts.

FIG. 15A is a bottom perspective view of a disposable probe assembly, in accordance with embodiments of the present inventive concepts.

FIG. 15B is a top perspective view of a base unit, in accordance with embodiments of the present inventive concepts.

FIGS. 16A-C are perspective views of a sterile barrier system, in accordance with embodiments of the present inventive concepts.

FIG. 17 is an exploded perspective view of a carrier for a disposable probe, in accordance with embodiments of the present inventive concepts.

FIGS. 17A-B are top and side views, respectively, of an insert, in accordance with embodiments of the present inventive concepts.

### DETAILED DESCRIPTION OF EMBODIMENTS

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

It will be further understood that when a first element is referred to as being “in”, “on”, “at” and/or “within” a second element, the first element can be positioned: within an internal space of the second element, within a portion of the second element (e.g. within a wall of the second element); positioned on an external and/or internal surface of the second element; and combinations of one or more of these, but is not limited thereto.

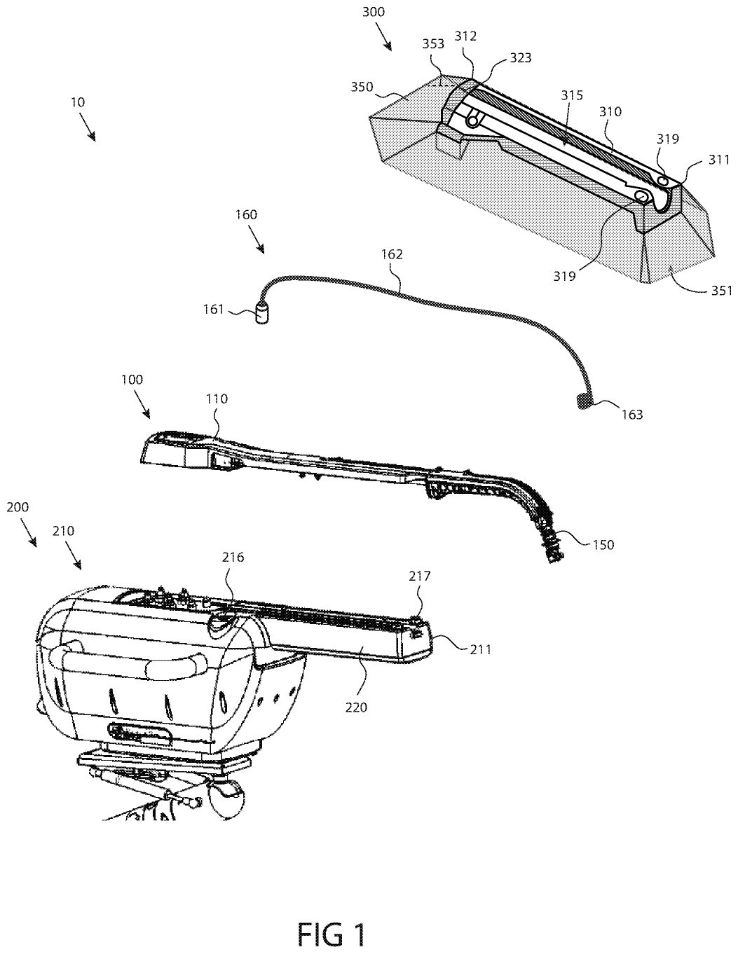

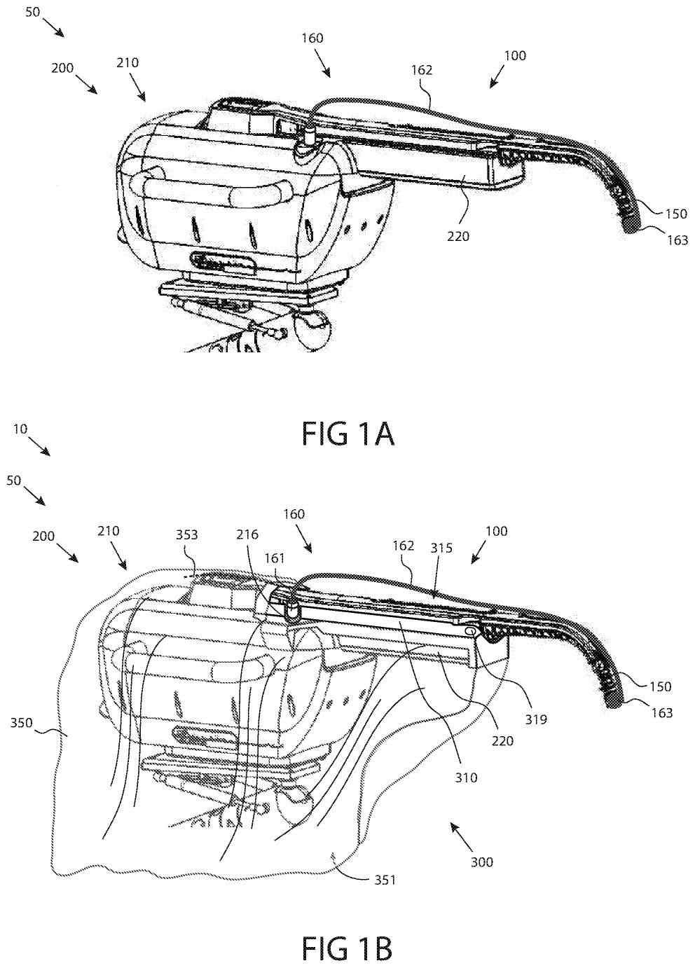

FIG. 1 is a perspective view of a robotic introducer system 10 comprising disposable probe assembly 100 and a sterile barrier system 300 separate from a base unit 200, in accordance with some embodiments. Disposable probe assembly 100 includes a housing 110, surrounding at least a portion of an articulating probe 150. Articulating probe 150 can be configured to extend, or otherwise be “fed” from housing 110, for example, a distal end of housing 110 as shown and described herein. Disposable probe assembly 100 can include a separate disposable or reusable camera assembly 160, including connector 161, cable 162, and camera 163, which is removably coupled to probe assembly 100. In alternate embodiments, camera 163 can be integral to articulating probe 150.

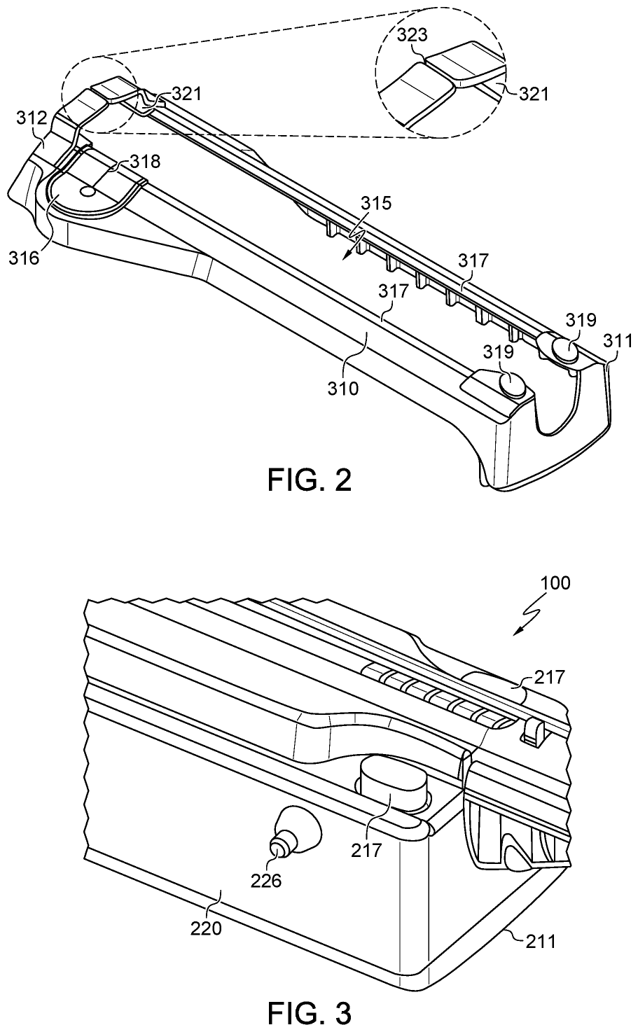

Sterile barrier system 300 can include a sterile frame 310 and a sterile drape 350. FIG. 2 is a perspective view of a sterile frame 310, with the sterile drape 350 removed for illustrative clarity. FIG. 1A is a perspective view of an assembled robotic introducer assembly 50, comprising disposable probe assembly 100 operably attached to base unit 200, such that one or more control forces, control energy, and/or data can be transferred between base unit 200 and disposable probe assembly 100. For example, base unit 200 can provide a force to steer, advance and/or otherwise manipulate articulating probe 150. In some embodiments, base unit 200 and disposable probe assembly 100, including articulating probe 150, are constructed and arranged as described in reference to PCT Application No. PCT/US 16/28374 filed Apr. 20, 2016 entitled “ARTICULATED ROBOTIC PROBES, SYSTEMS AND METHODS INCORPORATING THE SAME, AND METHODS FOR PERFORMING SURGICAL PROCEDURES, the contents of which are incorporated herein by reference in their entirety. FIG. 1B is a perspective view of the disposable probe assembly 100 operably attached to base unit 200, with a portion (e.g. a non-sterile portion) of the assembly covered by sterile barrier system 300, shown partially transparent. The disposable probe assembly 100, base unit 200, and sterile barrier system 300 collectively form the robotic introducer system 10.

The disposable probe assembly 100 is constructed and arranged to be used in one or more medical procedures, but fewer times than the base unit 200. Similarly, the sterile barrier system 300 is constructed and arranged to be used in one or more medical procedures, but fewer times than the base unit 200.

The term “use” can refer to a use of the disposable probe assembly 100, the base unit 200, and/or sterile barrier system 300 in one or more procedures for a particular patient. For example, the disposable probe assembly 100 can be used to perform one or more medical procedures on one patient, removed from the base unit 200, and replaced with a different disposable probe assembly 100 that is used to perform one or more medical procedures on a different patient. In another example, the disposable probe assembly 100 can be used to perform a procedure on one patient, removed from the base unit 200, and replaced with a different disposable probe assembly 100 that is used to perform a different procedure on the same patient. In some embodiments, multiple sterile barrier systems 300 are used on a single patient, in a single procedure, such as when probe assembly 100 is removed from base unit 200 in a single procedure (e.g. when probe assembly 100 is repositioned or a second probe assembly 100 is attached).

The base unit 200 is typically sanitized (e.g. cleaned, disinfected and/or sterilized) for each use (e.g. at least just prior to each use). One or more portions of base unit 200 are covered by the sterile barrier system 300. In some embodiments, the disposable probe assembly 100 is sanitized, typically sterilized, for a single use, and is removed from the base unit 200 and disposed of, after its single use.

The base unit 200 includes a proximal base portion 210, and a portion extending distally (i.e. towards the patient) from the proximal portion 210, base extension 220 with a distal end 211. Base unit can further include a connection port 216. Base unit 200 is constructed and arranged for reuse after a medical procedure, and is typically sanitized (e.g. cleaned but not sterilized) after each use.

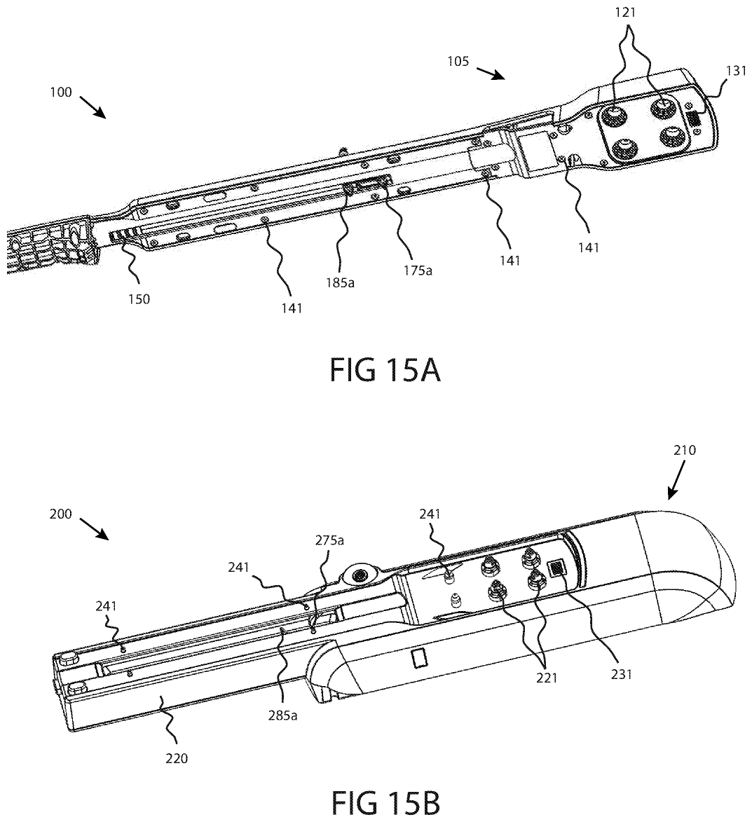

In some embodiments, the base unit 200 includes motors, gears, cables, circuitry, guide rails, motion transfer components, and/or other mechanical or electrical devices that communicate with disposable probe assembly 100 to control a movement of the articulating probe 150, and/or one or more tools in communication with the articulating probe 150. For example, the base unit 200 can comprise a motor, cable control assembly, or the like (not shown) that drives a carriage assembly within (not shown) the base extension 220, which in turn controls a movement of the articulating probe 150. For example, the base extension 220 can include a plurality of fingers or the like for translating (i.e. advancing and/or retracting) the articulating probe 150. The base unit 200 can include other elements similar to those described in PCT Application No. PCT/US 16/28374 filed Apr. 20, 2016 entitled “ARTICULATED ROBOTIC PROBES, SYSTEMS AND METHODS INCORPORATING THE SAME, AND METHODS FOR PERFORMING SURGICAL PROCEDURES, the contents of which are incorporated herein by reference in their entirety. Base unit 200 can be constructed and arranged as described in reference to FIGS. 15A and B herebelow.

As shown in FIG. 1A, robotic introducer assembly 50 comprises disposable probe assembly 100 removably attached to at least one of the proximal base portion 210 and/or base extension 220. The robotic introducer assembly 50 and the sterile barrier system 300 collectively comprise the robotic introducer system 10 shown in FIG. 1B. In some embodiments, robotic introducer assembly 50 further comprises camera assembly 160 removably attached to disposable probe assembly 100 and/or operably attached to base unit 200. Alternatively, camera assembly 160 can be integral to disposable probe assembly 100 (e.g. and disposed of with the disposal of probe assembly 100). In some embodiments, the disposable probe assembly 100 includes an articulating probe 150 comprising a link assembly (not shown) including an inner link mechanism comprising a plurality of inner links, and an outer link mechanism comprising a plurality of outer links, as described in connection with various embodiments herein. The link assembly is constructed and arranged to facilitate a manipulation of the articulating probe 150, which in turn can guide one or more surgical tools during a medical procedure. For example, the links can be constructed and arranged to form at least one multi-link inner probe (not shown) and a multi-link outer probe, similar to a probe assembly described in PCT Application No. PCT Application No. PCT/US 16/28374 filed Apr. 20, 2016, the contents of which are incorporated herein by reference above.

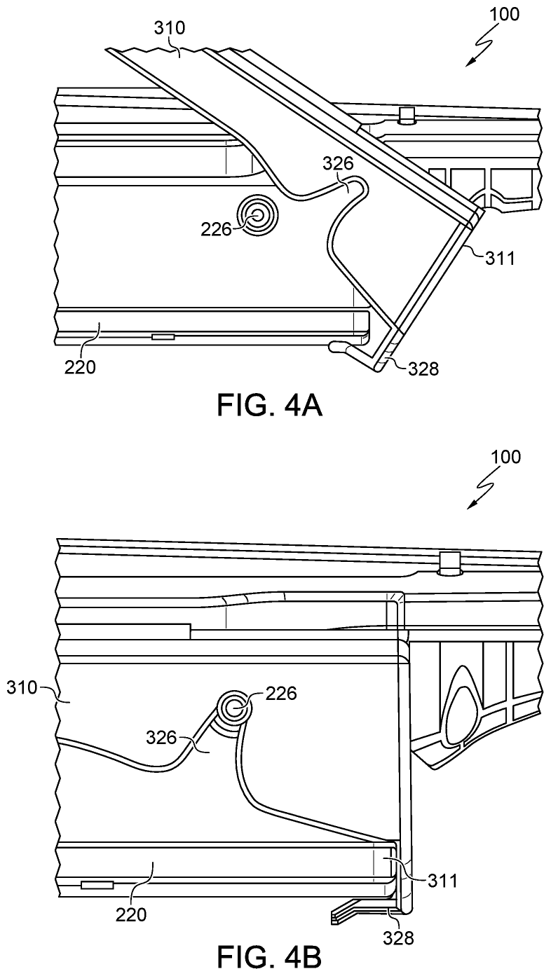

In some embodiments, the sterile barrier system 300 includes a sterile frame 310 and a sterile drape 350, each of which is sterile at the time of coupling to the base unit 200 and/or disposable probe assembly 100. In some embodiments, as shown in FIG. 1B, the sterile frame 310 is constructed and arranged to couple to the robotic introducer assembly 50. For example, sterile frame 310 can be constructed and arranged to couple to a portion of proximal base portion 210 and/or a proximal or mid portion of disposable probe assembly 100, as described herebelow in reference to FIG. 2, and to a portion of base extension 220, as shown in FIGS. 4A and 4B. The sterile frame 310 can have a single piece construction, i.e., molded or machined of a single unitary continuous material, or it can be assembled from multiple molded and/or machined components.

The sterile frame 310 has a first or distal end 311 that is configured for coupling to a distal end 211 of the base extension 220 (described below in reference to in FIGS. 4A and 4B) and a second or proximal end 312 that is configured for coupling to the proximal base portion 210 and/or a proximal or mid portion of disposable probe assembly 100 (described herebelow in reference to in FIGS. 2, 4A, and 4B). The sterile frame 310 includes an opening 315 between the first end 311 and the second end 312 that is constructed and arranged to receive at least a portion of the articulating probe 150 and/or a portion of disposable probe assembly 100 during assembly (e.g. placement of sterile barrier system 300 on robotic introducer assembly 50), and to expose at least a portion of the articulating probe 150 during operation of the system. As shown in FIG. 1B, the opening 315 fits over the assembled robotic introducer assembly 50 (i.e. the disposable probe assembly 100 is coupled to the base unit 200). The sterile frame 310 can include a covering 317 (shown in FIG. 2) or the like formed of an elastomeric material positioned about some or all of the opening 315 of the sterile frame 310 between the first end 311 and the second end 312 that can conform to at least a portion of disposable probe assembly 100. The covering 317 can serve as a gasket, seal, other interface between the disposable probe assembly 100 and the sterile frame 310.

The sterile frame 310 can include a grommet 316 or the like surrounding at least a portion of an opening in the sterile frame 310, configured to receive and maintain a sterile seal about a cable and/or a connector or portion thereof, for example, a portion of camera assembly 160, such as camera cable 162 and/or connector 161, operably connected to connection port 216 of base unit 200. Camera cable 162 or the like can extend from the base unit 200 through the grommet 316, to a tool port (not shown) or other location of, or otherwise attached, to the articulating probe 150.

A portion of the elastomeric material of covering 317 can comprise grommet 316, and can include a slit 318 extending between the opening of grommet 316 and the frame opening 315, such that a cable and/or connector (e.g. cable 162 and/or connector 161) can pass laterally from opening 315 into grommet 316 (e.g. while the cable is connected at both ends to portions of robotic introducer assembly 50).

In some embodiments, elastomeric material of covering 317 can comprise one or more portions, regions 319, configured to provide a sterile interface with and/or conform to one or more user depressible buttons of robotic introducer assembly 50. For example, base unit 200 can comprise one or more buttons, quick release buttons 217 (see FIG. 3), near the distal end 211 of the base extension 220, configured to release disposable probe assembly 100 from base unit 200, as described herein. The quick release buttons 217, also referred to as emergency release buttons, are actuated by depressing the elastomeric material of covering 317 which in turn applies a force to the button 217, which disconnects the disposable probe assembly 100 from the base unit 200.

In some embodiments, the second end 312 of the sterile frame 310 is positioned over a mid-portion of disposable probe assembly 100, and includes a relief feature 323, also referred to as a breakaway relief. that allows an expedient and safe removal of the disposable probe assembly 100 from the base unit 200 while allowing the sterile barrier system 300 to continue to cover the base unit 200. Accordingly, the sterile barrier system 300 can continue to separate a doctor or other sterile operator from the base unit 200, and/or other non-sterile components. For example, in an emergency, there may be a need for the operator to remove the disposable probe assembly 100 from the patient's mouth or other body orifice. The relief feature 323 can include a slit, opening, hinge, or a weakened or thinner portion, or a different material of the sterile frame 310 so that the disposable probe assembly 100 can be quickly and safely uncoupled from the base unit (e.g. by actuating the quick release buttons 217 at a distal end of the base extension 220). In doing so, the distal end of the disposable probe assembly 100 is separated from the base unit 200. The operator may pull the distal end of the disposable probe assembly 100 and in doing so, the disposable probe assembly 100 is removed from beneath sterile barrier system 300 through the frame opening 315 and relief feature 323 to complete the removal of the disposable probe assembly 100 from base unit 200. In these embodiments, the sterile barrier system 300 (including sterile drape 350 and/or sterile frame 310) are not removed but remain positioned over the at least a portion of base unit 200. As previously described, the probe assembly 100 can include a link assembly that is constructed and arranged to facilitate a manipulation of the articulating probe 150. In some embodiments, a proximal end of the probe assembly 150 can extend under the relief feature 323. However, in these embodiments, the link assembly extends from a distal end of the probe assembly 150 and does not extend to the relief feature 323 of the sterile barrier frame 310.

As shown in FIGS. 4A and 4B, the sterile frame 310 in some embodiments includes at its distal end 311 a notch 326, or the like, on one or both sides of the sterile frame 310 and a clip or other securing element, retention portion 328, for coupling to the base extension 220. The base extension 220 can include a guide pin 226 on one or both sides of base extension 220 that mates with the notch 326 of base extension 220. The retention portion 328 includes a lip or the like for locking the sterile frame 310 in place to prevent accidental dislodging. Also, the guide pin 226 and retention portion 328 can act as a guide for the sterile frame 310 to promote proper alignment and preventing potential contamination of the sterile field. Additionally, the retention portion 328 and/or notch 326 can be configured to provide tactile and/or audible feedback to a user to indicate proper attachment of sterile frame 310 to robotic introducer assembly 50.

As shown in FIG. 2, the proximal end 312 of the sterile frame 310 can include one or more projections or the like, engagement portions 321 for coupling to a portion of the disposable probe assembly 100 and/or base unit 200.

In some embodiments, the sterile drape 350 can comprise a high-density polyethylene (HDPE) or other flexible sterilized material. As described herein, a sterile barrier system 300 is provided during a procedure, to maintain sterility of the sterile environment by shielding the sterile environment (e.g. sterile operators, the patient, and/or sterile tools and the like) from non-sterile portions of robotic introducer system 10 (e.g. base unit 200). The sterile drape 350 is coupled to the perimeter of the sterile frame 310, and is constructed and arranged to cover both the proximal base portion 210 and base extension 200, for example, shown in FIG. 1B. In some embodiments, the sterile drape 350 comprises a bag like structure with an opening, opening 351, at its proximal end. Sterile drape 350 further comprises a hole (e.g. opposite the opening) and the sterile frame 310 is positioned in the hole and provides a rigid periphery of the hole. Sterile drape 350 can include a perforation or other region of separation, perforation 353 that extends along a portion of sterile drape 350 in a same longitudinal direction of the sterile frame 310 between the frame opening 315 and the periphery of the sterile drape 350. When the disposable probe assembly 100 is removed, for example, in an emergency (e.g. as described herein), the disposable probe assembly 100 can be pulled through the opening 315 in the sterile frame 310, and in doing so, tear the sterile drape 350 along the perforation 353 at the proximal end 312 of the sterile frame 310. The perforation permits the tearing of the sterile drape 350 for quick removal of and prevents the sterile drape 350 from interfering with the removal of the disposable probe assembly 100.

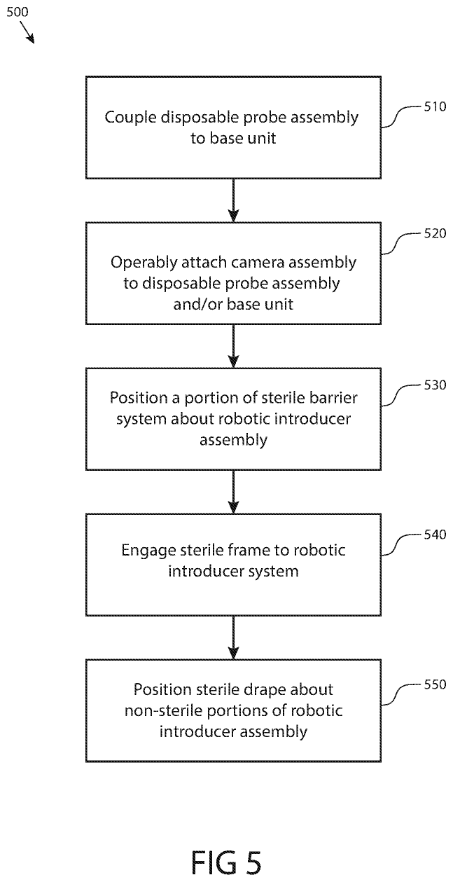

A robotic introducer assembly 50 can be assembled including the disposable probe assembly 100 and sterile barrier system 300 according to the following method 500 shown in FIG. 5.

In STEP 510, the disposable probe assembly 100 is coupled to the base unit 200. In some embodiments, disposable probe assembly 100 is coupled to base unit 200 at least in part along base extension 220, such that linear motion transfer from base unit 200 to disposable probe assembly 100 can occur along base extension 220.

In STEP 520, camera assembly 160 can be operably attached to base unit 200 and disposable probe assembly 100. Camera 163 can be fixedly attached to the distal portion of articulating probe 150 and camera cable 162 can be longitudinally aligned with (and in some embodiments secured to at least a portion of) disposable probe assembly 100. Camera connector 162 can be operably attached to port 216 of base unit 200.

In STEP 530, the distal end of robotic introducer assembly 50 (i.e. the distal portion of disposable probe assembly 100) is inserted into sterile drape 350 via opening 351 (e.g. when drape 350 comprises a bag like construction) and exiting opening 315 of sterile frame 310, and distal end 311 of sterile frame 310 is positioned proximate (as shown in FIG. 4A) the distal end of base extension 220.

In STEP 540, sterile frame 310 is rotated about its distal end 311, or otherwise repositioned to align with and/or engage a mid-portion of disposable probe assembly 100 and/or proximal base portion 210. Notch 326 engages guide pin 226 and retention portion 328 engages the distal end of base extension 220 securing the distal end of sterile frame 310 to robotic introducer assembly 50 (as shown in FIG. 4B). In some embodiments, grommet 316 laterally or otherwise slidingly receives at least a portion of camera cable 162 and/or connector 161, creating a seal (e.g. a contamination preventing seal and/or sterile seal) about a portion of camera assembly 160.

In STEP 550, sterile drape 350 can be unfolded or otherwise repositioned to cover remaining non-sterile portions of base 200 and/or portions of system 10, for example stands, supports, or carts used for positioning base unit 200.

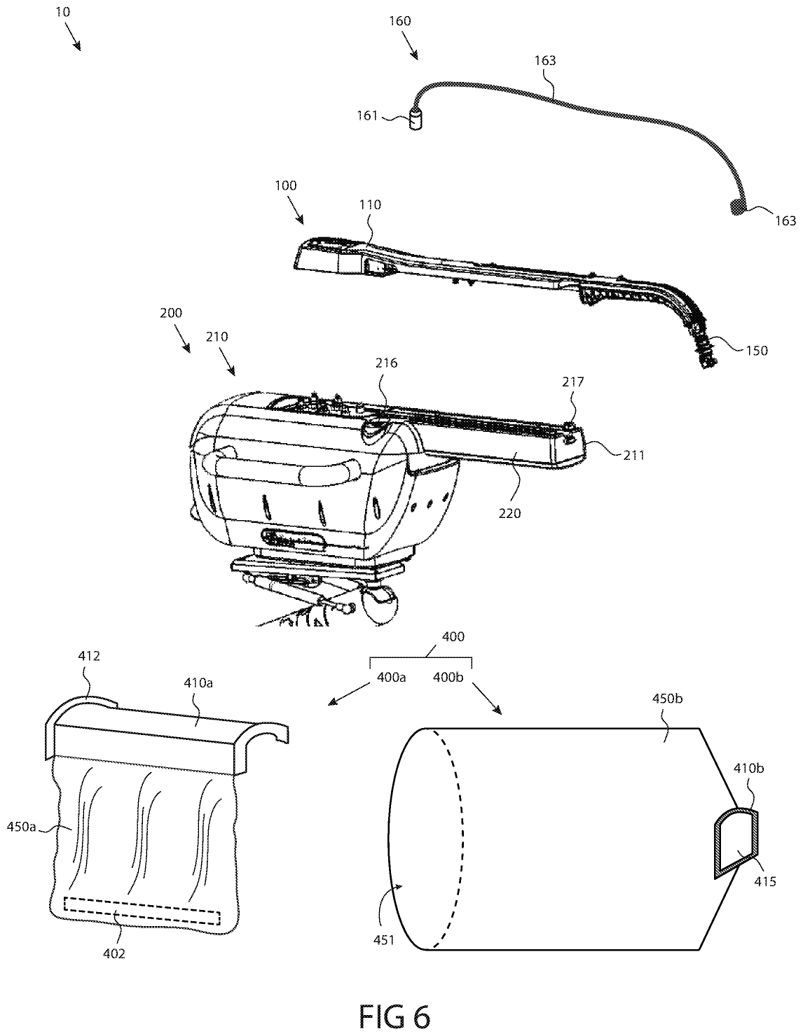

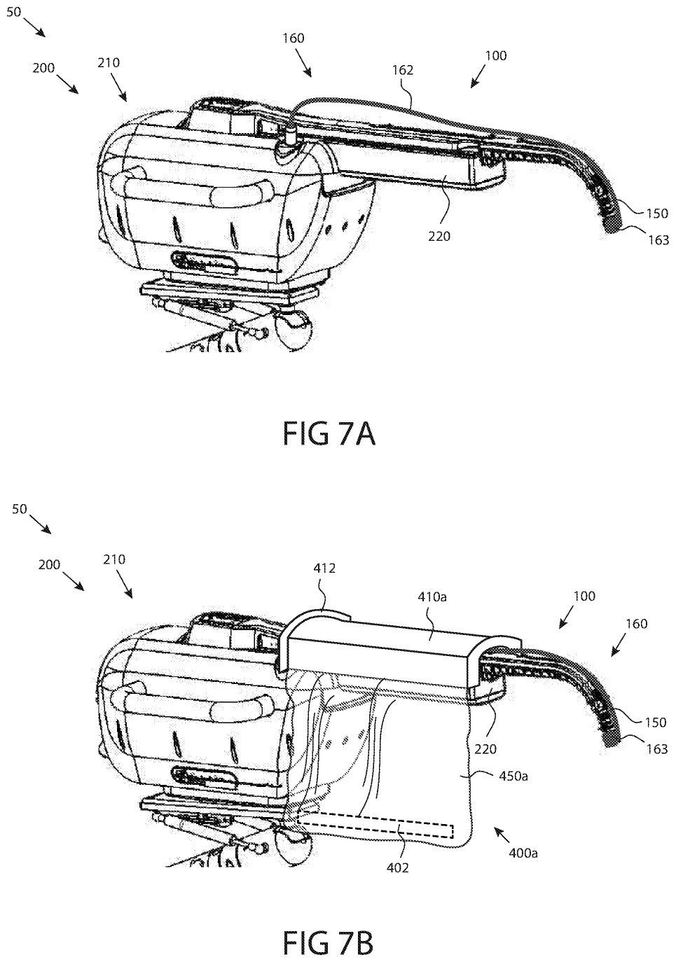

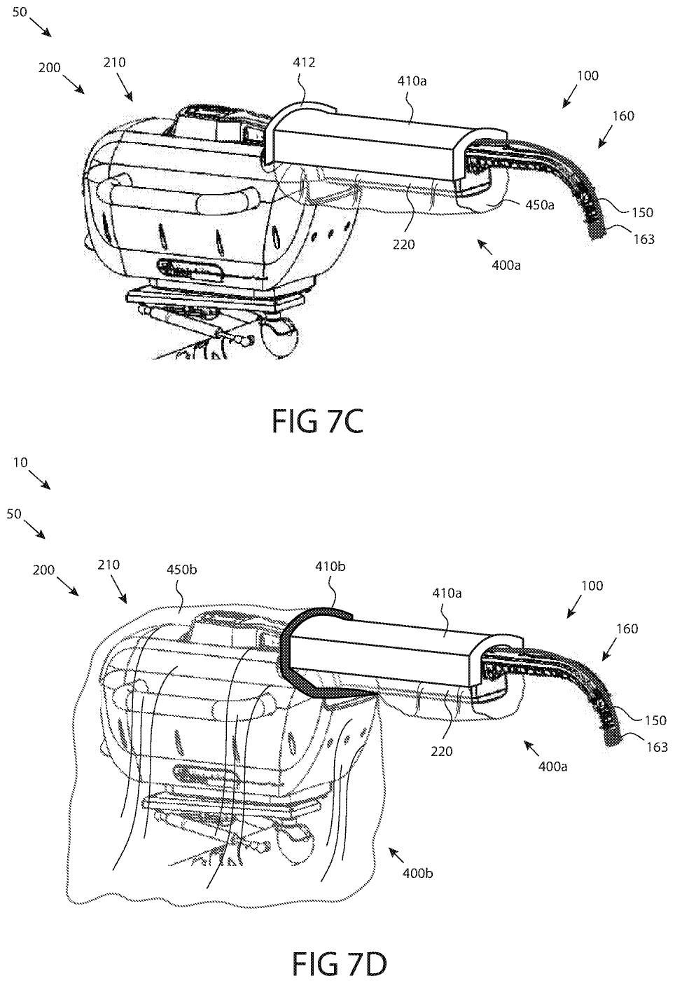

FIG. 6 is a perspective view of a disposable assembly 100 and a sterile barrier system 400 separate from a base unit 200, in accordance with other embodiments. FIGS. 7A-D are perspective views of the steps for assembling robotic introducer assembly 50, comprising attaching disposable probe assembly 100 to base unit 200, and the draping of the assembly with sterile barrier system 400. Robotic introducer assembly 50 can be of similar construction and arrangement as those described hereabove in reference to FIGS. 1-5. FIG. 7A is a perspective view of an assembled robotic introducer assembly 50, comprising disposable probe assembly 100 operably attached to base unit 200, such that one or more control forces can be transferred between base unit 200 and disposable probe assembly 100. FIG. 7B is a perspective view of robotic introducer assembly 50 with a first portion 400a of sterile barrier system 400, shown partially transparent, attached to a portion of the robotic introducer system 50, described herebelow. FIG. 7C is a perspective view of the robotic introducer assembly 50 with the first portion 400a further secured to the introducer assembly 50. FIG. 7D is a perspective view of robotic introducer assembly 50, partially draped by first portion 400a of sterile barrier system 400, and the remaining portion of introducer system 50 draped by a second portion 400b of sterile barrier system 400.

The sterile barrier system 400 includes a first portion 400a comprising a first sterile drape 450a coupled to a first frame 410a and a second portion 400b comprising a second sterile drape 450b coupled to a second sterile frame 410b constructed and arranged to interface with first sterile frame 410a. These portions 400a and 400b of the barrier system 400 are sterile and disposable. First sterile frame 410a comprises a projection 412, configured to interface with a portion of second sterile frame 410b, providing a seal (e.g. a contamination preventing seal and/or sterile seal) between the first and second portions 400a and 400b when draped on robotic introducer assembly 50 as described herebelow. The first sterile drape 450a extends from first sterile frame 410a and is configured to wrap around and cover a bottom region, or belly, of the base extension 200, and attach to the other side of the first sterile frame 410a via a fastening element 402 (i.e. tape, adhesive, Velcro™, or other coupling mechanism), when the first sterile frame 410a is mounted over the top portion of the base extension 220 over a portion of the disposable probe assembly 100 above the base extension 220, as shown in FIGS. 7B and 7C. The second drape 450b can comprise a bag like structure with an opening, opening 451, at its proximal end. The second sterile drape 450b further comprises a hole (e.g. opposite the opening 451) in a center region of sterile drape 450b that interfaces with the second sterile frame 410b. When the second portion 400b of sterile barrier system 400 is installed onto robotic introducer assembly 50, the distal portions of disposable probe assembly 100 and base extension 220 are fed into sterile drape 450b and out opening 415 of second sterile frame 410b. The second sterile frame 410b is positioned proximate proximal base portion 210 and directly interfaces with a projection 412 at the proximal end of the first sterile frame 410a. First portion 400a and second portion 400b can be constructed and arranged such that one or more regions of overlap between the two portions exist when the robotic introducer assembly 50 is fully draped by sterile barrier system 400, such as to limit contact and/or provide isolation between any non-sterile portion of robotic introducer assembly 50 and the sterile field or any sterile operators of system 10 (e.g. to prevent exposure of any non-sterile portion to the sterile field or sterile operators).

As shown in FIG. 7A, robotic introducer assembly 50 comprises disposable probe assembly 100 removably attached to at least one of the proximal base portion 210 and/or base extension 220. In some embodiments, robotic introducer assembly 50 further comprises camera assembly 160 removably attached to disposable probe assembly 100 and/or operably attached to base unit 200. In some embodiments, the disposable probe assembly 100 includes an articulating probe 150 as described hereabove in reference to FIG. 1A.

As shown in FIG. 7B, first sterile frame 410a is configured to cover a portion of robotic introducer assembly 50, including a portion of disposable probe assembly 100, base extension 220, and camera assembly 160, shielding non-sterile portions thereof from the sterile field. As shown in FIG. 7C, first sterile drape portion 450a is configured to wrap underneath base extension 220, fastening to the opposite side of first sterile frame 410a, further shielding non-sterile portions of introducer assembly from the sterile field. As shown in FIG. 7D, second portion 400b is configured to bag (i.e. drape to shield) the remaining unshielded non-sterile portions of introducer assembly 50 from the sterile field.

During removal, for example, in an emergency (e.g. as described herein), the first sterile frame 410a can include holes (not shown) for providing access to quick release buttons 217, which when pressed can permit the disposable probe assembly 100 to be separated from the base unit 200. The operator may pull the distal end of the disposable probe assembly 100 and in doing so, the force caused by the pulling motion separates the first sterile frame 410a from base extension 220. The first sterile frame 410a can be removed with the disposable probe assembly 100. The sterile drape 450b can remain positioned about the base unit 200. In some embodiments, second sterile frame 410b is configured to flex, break, or otherwise displace to allow first sterile frame 410a to disengage from base extension 220.

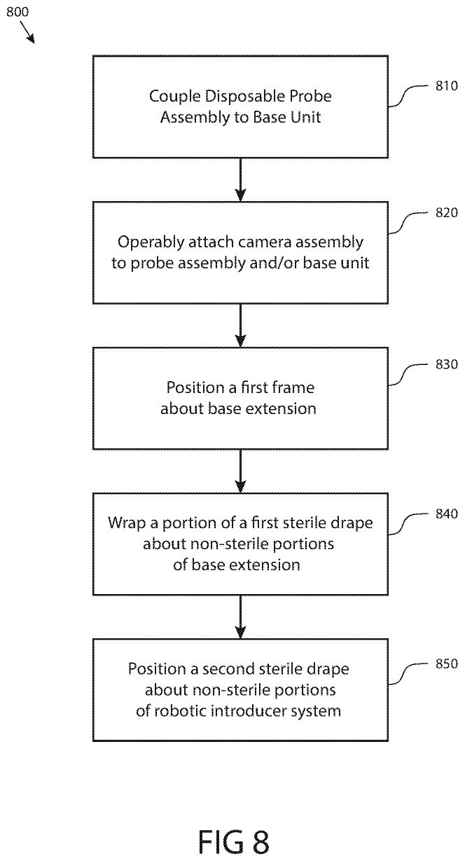

A robotic introducer assembly 50 can be assembled including the disposable probe assembly 100 and sterile barrier system 400 according to the following method 800 shown in FIG. 8.

In STEP 810, the disposable probe assembly 100 is coupled to the base unit 200. In some embodiments, disposable probe assembly 100 is coupled to base unit 200 at least in part along base extension 220, such that linear motion transfer from base unit 200 to disposable probe assembly 100 can occur along base extension 220.

In STEP 820, camera assembly 160 can be operably attached to base unit 200 and disposable probe assembly 100. Camera 163 can be fixedly attached to the distal portion of articulating probe 150 and camera cable 162 can be longitudinally aligned with (and in some embodiments secured to at least a portion of) disposable probe assembly 100. Camera connector 162 can be operably attached to port 216 of base unit 200.

In STEP 830, the first sterile frame 410a of sterile barrier system 400 is positioned over the top portion of the base extension 220, such that at least a portion of the robotic introducer assembly 50 is covered by first sterile frame 410a.

In STEP 840, the first sterile drape 450a extending from one side of the first sterile frame 410a is wrapped around the remaining non-sterile bottom portion of the base extension 220 and is attached to the opposite side of the first sterile frame 410a.

In STEP 850, the distal end of robotic introducer assembly 50 (i.e. the distal portion of disposable probe assembly 100) is inserted into a second sterile drape 450b via opening 451 (e.g. when sterile drape 450b comprises a bag like construction) and exiting opening 415 of the second sterile frame 410b of sterile barrier system 400. The second sterile frame 410b is positioned proximate (as shown in FIG. 7D) the distal end of base extension 220. Second sterile drape 450b can be unfolded or otherwise repositioned to cover remaining non-sterile portions of base 200 and/or portions of system 10, for example, stands, supports, or carts used for positioning base unit.

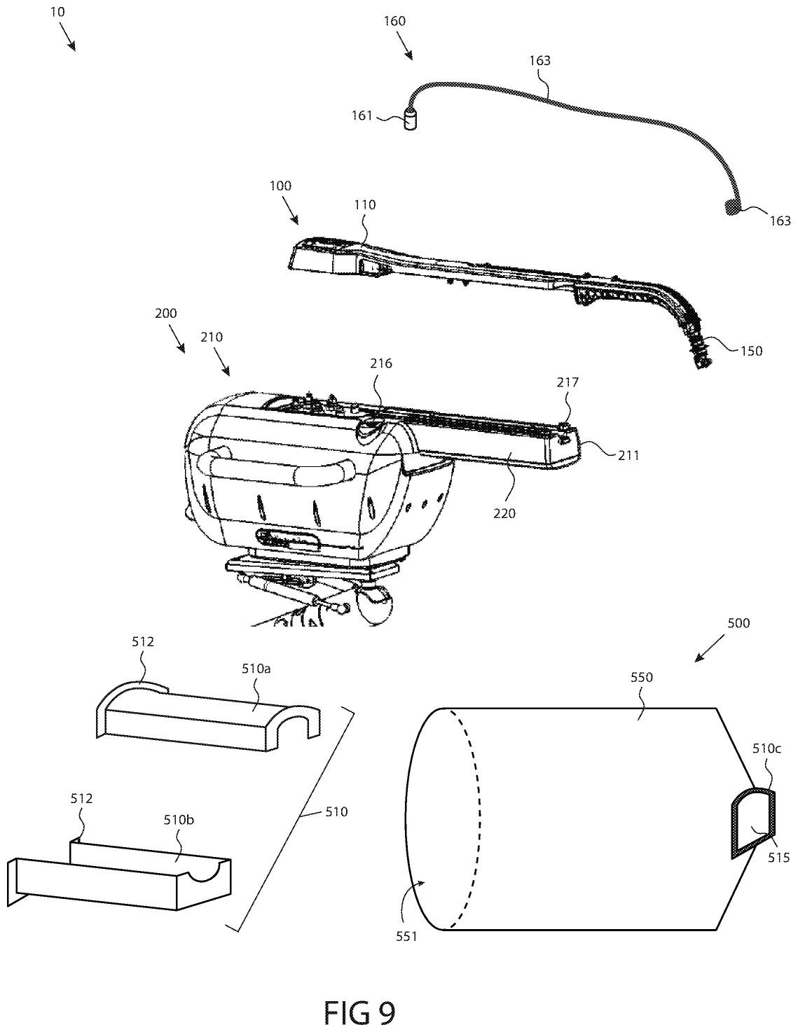

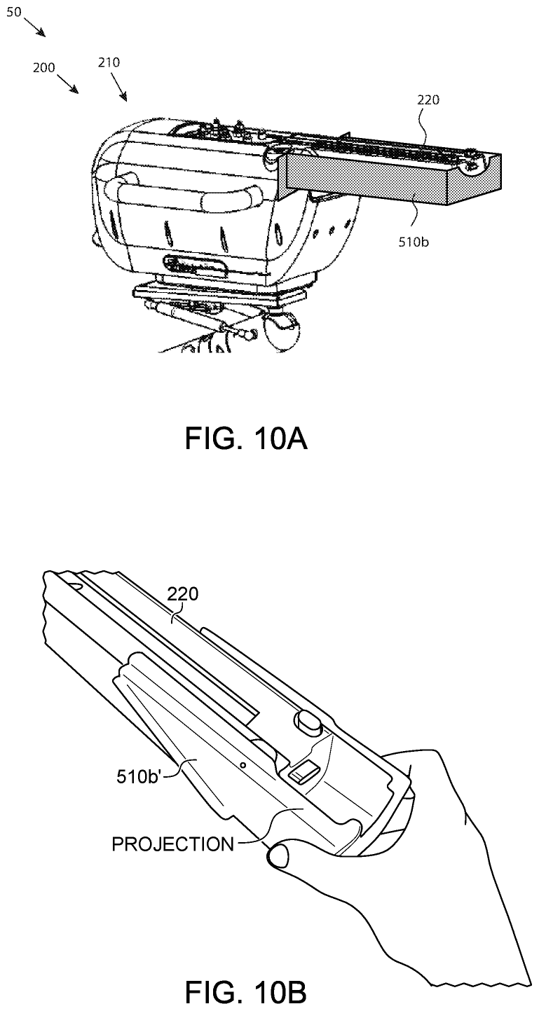

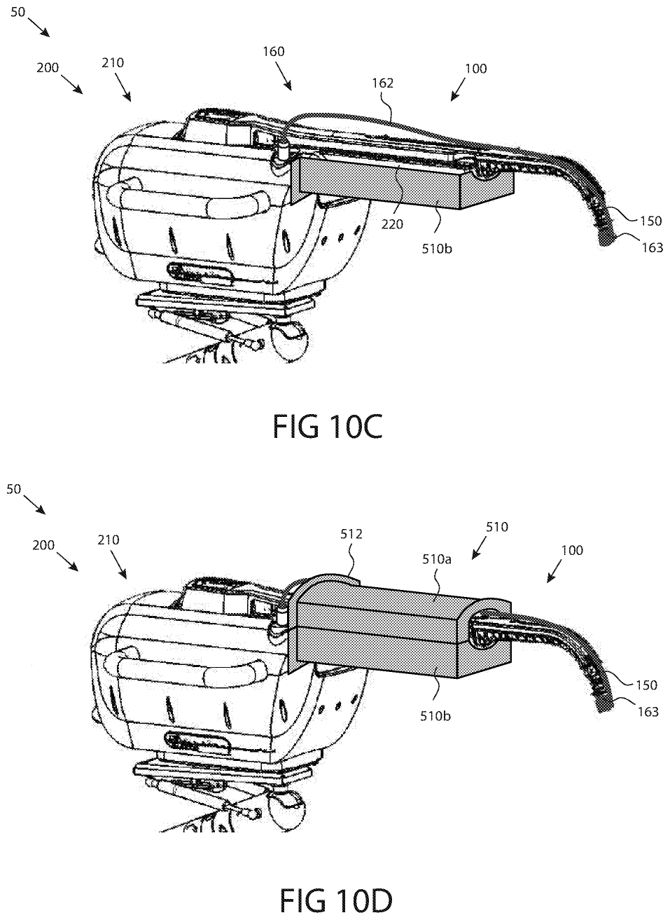

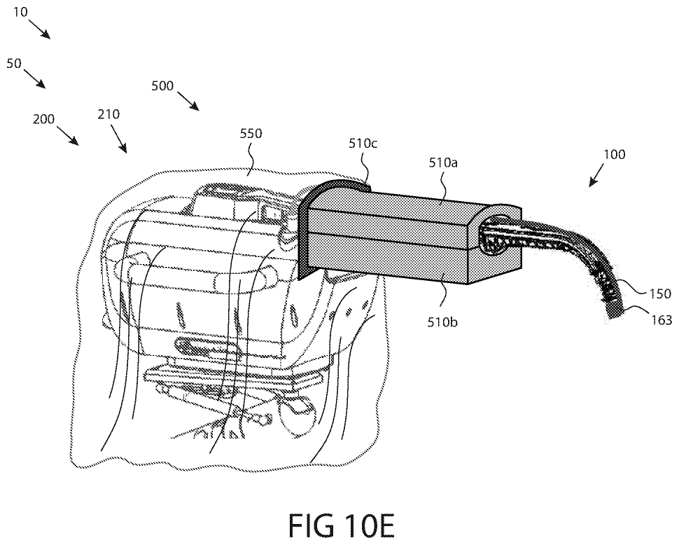

FIG. 9 is a perspective view of a disposable probe assembly 100 and a sterile barrier system 500 separate from base unit 200, in accordance with other embodiments. FIGS. 10A-E are perspective views of the steps for assembling robotic introducer assembly 50, comprising attaching disposable probe assembly 100 to base unit 200, and the draping of the assembly with sterile barrier system 500 of FIG. 9.

The disposable probe assembly 100 and robotic introducer assembly 50 can be similar to, or the same, as disposable probe assembly 100 and robotic introducer assembly 50 described and illustrated with respect to FIGS. 1-5. In particular, the robotic introducer system assembly 50 comprises a base unit 200 constructed and arranged to be reusable, and the disposable probe assembly 100, provided in a sterile condition for a single use, configured to be removably attached to the base unit 200.

The sterile barrier system 500 includes a multi-component sterile frame assembly 510, comprising an upper sterile frame 510a and a lower sterile frame 510b, and a single sterile drape 550, comprising a third sterile frame 510c with an opening 515. These elements 510, 550 of the sterile barrier system 500 are sterile and disposable.

The upper sterile frame 510a and lower sterile frame 510b of sterile frame assembly 510 are constructed and arranged for covering a forward section of the robotic introducer assembly 50, in particular, at least a portion of disposable probe assembly 100 and a portion of base extension 220. For example, sterile frame assembly 510 can cover a portion of base extension 220 remaining exposed to the sterile field after disposable probe assembly 100 is removably attached to base unit 200.

As shown in FIG. 10A, lower sterile frame 510b is configured to attach (e.g. to removably attach) to the underside of base extension 220. In some embodiments, the lower sterile frame 510b slides axially onto the base extension 220 and is held in place by a frictional engagement and/or a coupling mechanism for holding the lower sterile frame 510b against the base extension 220. In some embodiments, lower sterile frame 510b comprises one or more projections and/or slots configured to slidingly engage one or more slots and/or projections of base unit 200. As shown in FIG. 10B, a photograph illustrates how lower sterile frame 510b′ slides axially onto base extension 220 with the aid of projections to engage the distal portion of base extension 220.

As shown in FIG. 10C, disposable probe assembly 100 is removably attached to at least one of the proximal base portion 210 and/or base extension 220. Camera assembly 160 is removably attached to disposable probe assembly 100 and/or operably attached to base unit 200. In some embodiments, the disposable probe assembly 100 includes an articulating probe 150 as described hereabove in reference to FIG. 1A.

As shown in FIG. 10D, the upper sterile frame 510a is positioned on top of at least a portion of disposable probe assembly 100 and base extension 220, such that upper sterile frame 510a is coupled to the lower sterile frame 510b, creating a sterile barrier partially encapsulated by sterile frame assembly 510. The articulating probe 150 of disposable probe assembly 100 can translate within, and extend from, the probe assembly housing 110 without interference with sterile frame assembly 510. Sterile frame assembly 510 can be configured to cover non-sterile portions of base extension 220 from the sterile field. Sterile frame assembly 510 comprises a projection 512 (e.g. a single circumferential projection comprised of one or more projections of upper sterile frame 510a aligned with one or more projections of lower sterile frame 510b), configured to interface with a portion of third sterile frame 510c, providing a seal (e.g. a contamination preventing seal and/or sterile seal) between sterile frame assembly 510 and sterile drape 550 when draped on robotic introducer assembly 50 as described herebelow.

As shown in FIG. 10E, the sterile drape 550 is installed. The sterile drape 550 can comprise a bag like structure with an opening, opening 551, at its proximal end. The sterile drape 550 further comprises a hole, opening 515 (e.g. opposite the opening 551) in a center region of drape 550 that interfaces with the third sterile frame 510c. When the sterile drape 550 of sterile barrier system 500 is installed onto robotic introducer assembly 50, the distal portions of disposable probe assembly 100 and base extension 220 are fed into sterile drape 550 and out opening 515 of third sterile frame 510c. The third sterile frame 510c is positioned proximate proximal base portion 210 and directly interfaces with projection 512 at the proximal end of the sterile frame assembly 510. Sterile frame assembly 510 and sterile drape 550 can be constructed and arranged such that one or more regions of overlap between the two elements exist when the robotic introducer assembly 50 is fully draped by sterile barrier system 500, such as to limit contact and/or provide isolation between any non-sterile portion of robotic introducer assembly 50 and the sterile field or any sterile operators of system 10 (e.g. to prevent exposure of any non-sterile portion to the sterile field or sterile operators).

During removal, for example, in an emergency (e.g. as described herein), the upper sterile frame 510a can include holes for providing access to quick release buttons 217, which when pressed can permit the disposable probe assembly 100 to be separated from the base unit 200. The operator may pull the distal end of the disposable probe assembly 100 and in doing so, the force caused by the pulling motion separates the sterile frame assembly 510. The upper sterile frame 510a can be removed with the disposable probe assembly 100. The sterile drape 550 and lower sterile frame 510b can remain positioned about the at least a portion of base unit 200 (as shown in FIG. 10A). In some embodiments, sterile frame 510c is configured to flex, break, or otherwise displace to allow upper sterile frame 510a to disengage from sterile frame 510c.

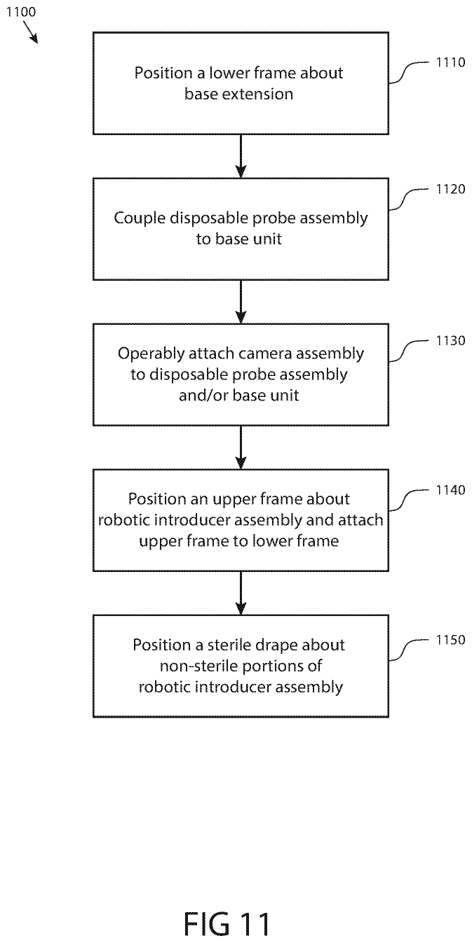

A robotic introducer assembly 50 can be assembled including the disposable probe assembly 100 and sterile barrier system 500 according to the following method 1100 shown in FIG. 11.

In STEP 1110, the lower sterile frame 510b of sterile barrier system 500 is positioned under the bottom portion of the base extension 220, such that at least a portion of the base extension 220 is covered by lower sterile frame 510b.

In STEP 1120, the disposable probe assembly 100 is coupled to the base unit 200. In some embodiments, disposable probe assembly 100 is coupled to base unit 200 at least in part along base extension 220, such that linear motion transfer from base unit 200 to disposable probe assembly 100 can occur along base extension 220.

In STEP 1130, camera assembly 160 can be operably attached to base unit 200 and disposable probe assembly 100. Camera 163 can be fixedly attached to the distal portion of articulating probe 150 and camera cable 162 can be longitudinally aligned with (and in some embodiments secured to at least a portion of) disposable probe assembly 100. Camera connector 162 can be operably attached to port 216 of base unit 200.

In STEP 1140, the upper sterile frame 510a of sterile barrier system 500 is positioned over the top portion of the base extension 220, such that at least a portion of the robotic introducer assembly 50 is covered by upper sterile frame 510b. Upper sterile frame 510a is coupled to lower sterile frame 510b.

In STEP 1150, the distal end of robotic introducer assembly 50 (i.e. the distal portion of disposable probe assembly 100) is inserted into sterile drape 550 via opening 551 (e.g. when sterile drape 550 comprises a bag like construction) and exiting opening 515 of the third sterile frame 510c of sterile barrier system 500. The third sterile frame 510c is positioned proximate (as shown in FIG. 10E) the distal end of base extension 220. Sterile drape 550 can be unfolded or otherwise repositioned to cover remaining non-sterile portions of base unit 200 and/or portions of system 10, for example, stands, supports, or carts used for positioning base unit 200.

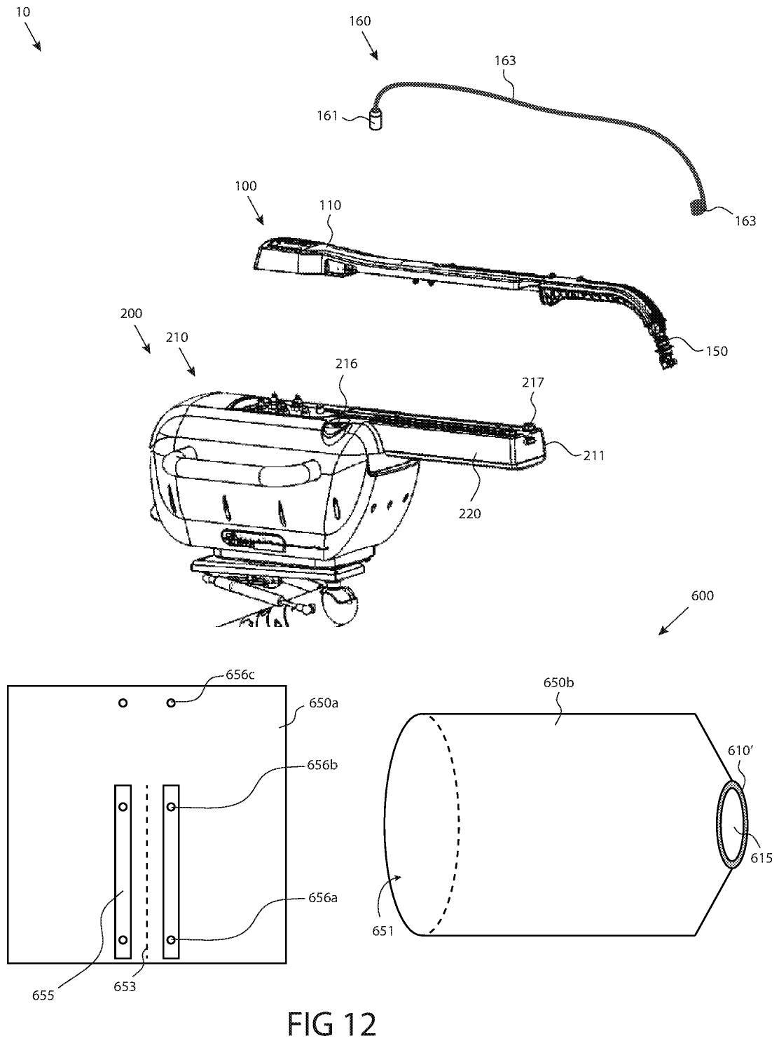

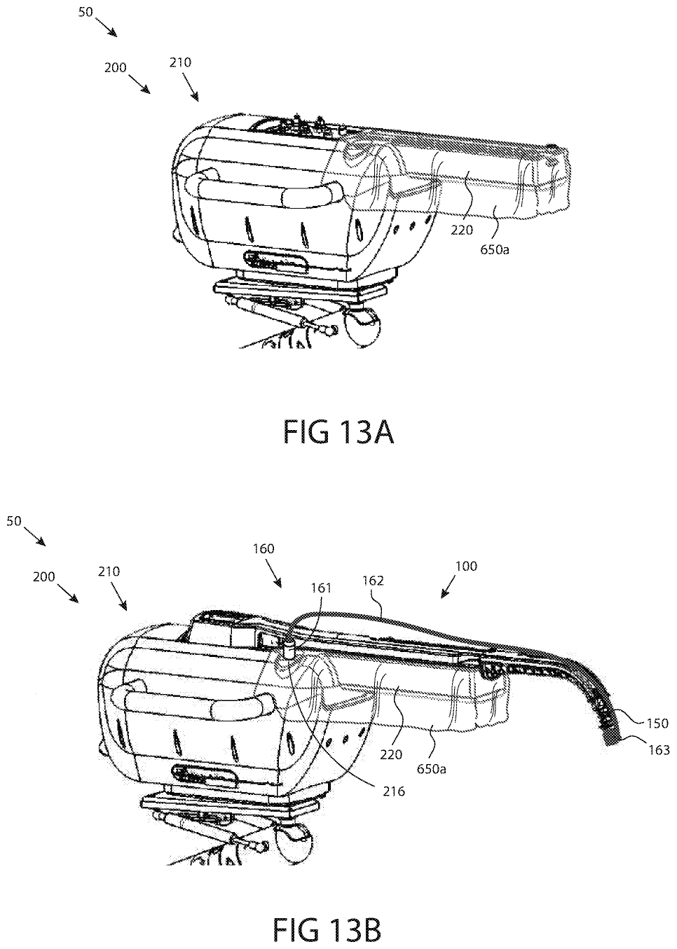

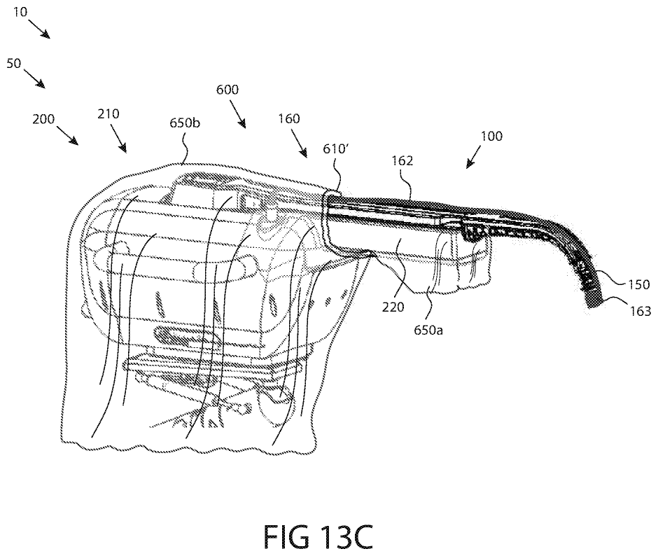

FIG. 12 is a perspective view of a disposable probe assembly 100 and a sterile barrier system 600 separate from base unit 200, in accordance with other embodiments. FIG. 13A-13C are perspective views of the steps for assembling robotic introducer assembly 50, comprising attaching disposable probe assembly 100 to base unit 200, and the draping of the assembly with sterile barrier system 600 of FIG. 12.

The disposable probe assembly 100 and robotic introducer assembly 50 can be similar to or the same as disposable probe assembly 100 and robotic introducer system 50 described and illustrated with respect to FIGS. 1-5. Details thereof are therefore omitted due to brevity. In particular, the robotic introducer assembly 50 comprises a base unit 200 constructed and arranged to be reusable, and the disposable probe assembly 100 provided in a sterile condition for a single use, configured to be attached to the base unit 200.

The sterile barrier system 600 includes a first sterile drape 650a and a second sterile drape 650b. The first sterile drape 650a can be constructed and arranged as a sheet of sterile material such as sterile plastic and/or sterile drape material. First sterile drape 650a can comprise one or more stiffening elements 655 configured to provide a rigidity to drape 650a to assist a user in affixing first sterile drape 650a to base unit 200 as described herebelow. First sterile drape 650a can further comprise one or more alignment holes, holes 656a, 656b, and 656c shown, configured to slidingly engage one or more alignment pins 241 (not shown) of base unit 200 to assist a user in affixing first sterile drape 650a to base unit 200 as described herebelow. The second sterile drape 650b can comprise a bag like structure with an opening, opening 651, at its proximal end. The second sterile drape 650b further comprises a hole or opening 615 (e.g. opposite the opening 651) in a center region of second sterile drape 650b, surrounded by an elastic portion, cinch 610′, comprising opening 615.

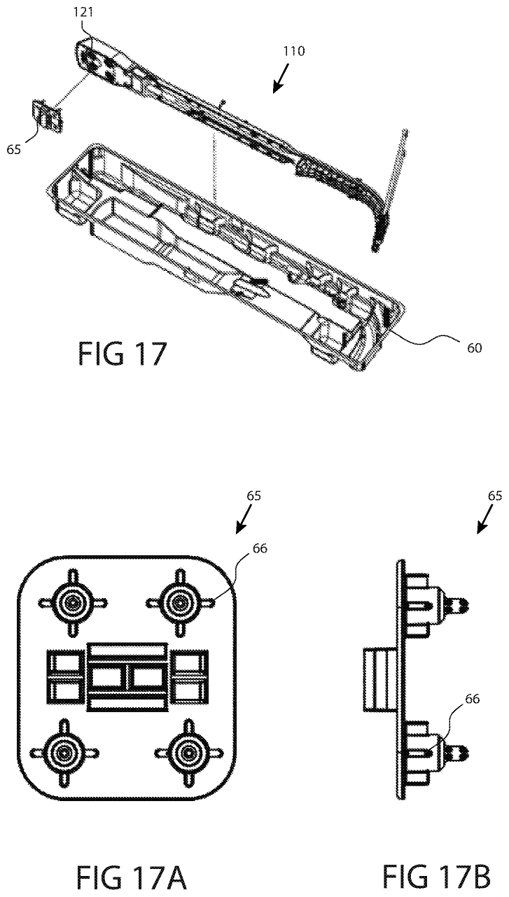

As shown in FIG. 13A, the first sterile drape 650a is positioned about at least a portion of base unit 200, for example base extension 220 as shown. The first sterile drape 650a includes a perforation 653, or slit or other opening region of separation that is positioned on the top of the base extension 220 and that extends in a longitudinal direction of the extension 220. The perforation 653 can extend along the center region of the first sterile drape 650a in a same longitudinal direction as stiffening elements 655, such that precise placement of perforation 653 can be achieved. For example, perforation 653 can be aligned with a channel in base extension 220 configured to receive one or more projections from disposable probe assembly 100, for the longitudinal control of articulating probe 150 by base unit 200, as described herebelow in reference to FIGS. 17A and 17B. In some embodiments, the alignment holes 656a can slidingly receive one or more alignment pins 241 of base unit 200 near the distal end of base extension 220, holes 656b can receive one or more alignment pins 241 of base unit 200 near the distal end of proximal base portion 210, and holes 656c can receive one or more alignment pins 241 of base unit 200 proximal to base extension 220, such as to properly align the first sterile drape with base unit 200 prior to removably attaching disposable probe assembly 100 to base unit 200 over first sterile drape 650a. As shown in FIG. 12, one or more alignment holes 656 can be integral to stiffening elements 655, such as to properly align the elements 655 as well as sterile drape 650a.

As shown in FIG. 13B, the disposable probe assembly 100 is attached to at least one of the proximal base portion 210 and/or base extension 220, over at least a portion of first drape 650a (i.e. at least a portion of sterile drape 650a is between disposable probe assembly 100 and base unit 200). In doing so, one or more elements of the disposable probe assembly 100, such as coupling mechanisms, bobbins, guide pins, and so on, interface with counterpart elements of the base unit 200 through the perforation 653 as described herein. In some embodiments, the disposable probe assembly 100 includes an articulating probe 150 as described hereabove in reference to FIG. 1A. In some embodiments, first sterile drape 650a can be tied, cinched, or otherwise secured beneath base extension 220.

One or more external connections can be made after the disposable probe assembly 100 is attached to the base unit 200. For example, camera assembly 160 is removably attached to disposable probe assembly 100 and/or operably attached to base unit 200, as shown. Connector 161 can be operably attached to connector 216 of base unit 200 around or through a portion (e.g. an opening or cutaway) of sterile drape 650a.

As shown in FIG. 13C, the second sterile drape 650b is positioned about robotic introducer assembly 50. The distal portions of disposable probe assembly 100 and base extension 220 are fed into sterile drape 650b and out opening 615 of cinch 610′. The cinch 610′ is positioned proximate proximal base portion 210 and elastically engages with base extension 220. First sterile drape 650a and second sterile drape 650b can be constructed and arranged such that one or more regions of overlap between the two sterile drapes exist when the robotic introducer assembly 50 is fully draped by sterile barrier system 600, such as to limit contact and/or provide isolation between any non-sterile portion of robotic introducer assembly 50 and the sterile field or any sterile operators of system 10 (e.g to prevent exposure of any non-sterile portion to the sterile field or sterile operators).

During removal, for example, in an emergency (e.g. as described herein), the first sterile drape 650a can include holes and/or a conformable portion for providing access to quick release buttons 217, which when pressed can permit the disposable probe assembly 100 to be separated from the base unit 200. The operator may pull the distal end of the disposable probe assembly 100 and in doing so, the force caused by the pulling motion overcomes the elastic force of cinch 610′, and disposable probe assembly 100 can be removed from base unit 200. The sterile drape 650a and 650b can remain positioned about at least a portion of base unit 200.

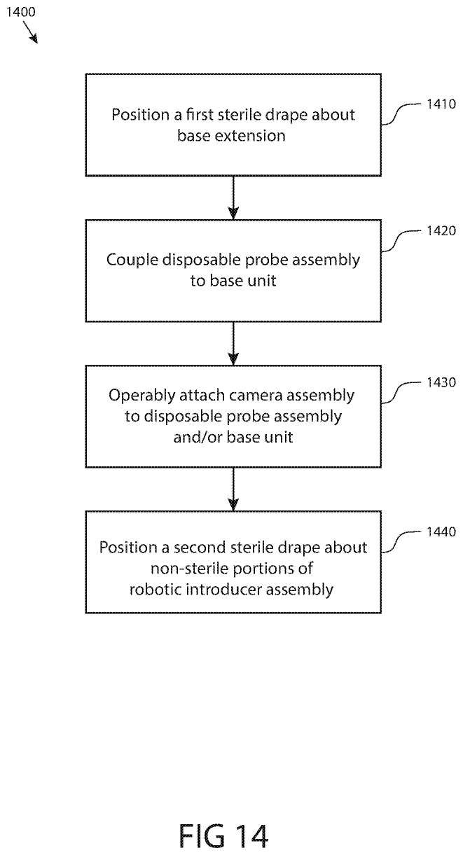

A robotic introducer assembly 50 can be assembled including the disposable probe assembly 100 and sterile barrier system 600 according to the following method 1400 shown in FIG. 14.

In STEP 1410, the first sterile drape 650a of sterile barrier system 600 is positioned over the top portion of the base extension 220, such that at least a portion of base extension 220 is covered by first sterile drape 650a. In some embodiments, alignment holes 656a-c of first sterile drape 650a are positioned to slidingly receive alignment pins of base extension 220. Additionally or alternatively, stiffening elements 655 are positioned along a top portion of base extension 220 to align perforation 653 with a channel of base extension 220.

In STEP 1420, the disposable probe assembly 100 is coupled to the base unit 200. In some embodiments, disposable probe assembly 100 is coupled to base unit 200 at least in part along base extension 220, such that linear motion transfer from base unit 200 to disposable probe assembly 100 can occur along base extension 220.

In STEP 1430, camera assembly 160 can be operably attached to base unit 200 and disposable probe assembly 100. Camera 163 can be fixedly attached to the distal portion of articulating probe 150 and camera cable 162 can be longitudinally aligned with (and in some embodiments, secured to at least a portion of) disposable probe assembly 100. Camera connector 162 can be operably attached to port 216 of base unit 200.

In STEP 1440, the distal end of robotic introducer assembly 50 (i.e. the distal portion of disposable probe assembly 100) is inserted into second sterile drape 650b via opening 651 (e.g. when second sterile drape 650b comprises a bag like construction) and exiting opening 615 of the cinch 610′ of sterile barrier system 600. The cinch 610′ is positioned proximate (as shown in FIG. 13C) the distal end of base extension 220. Second sterile drape 650b can be unfolded or otherwise repositioned to cover remaining non-sterile portions of the base unit 200 and/or portions of system 10, for example, stands, supports, or carts used for positioning base unit 200.

FIG. 15A is a bottom perspective view of the disposable probe assembly 100, in accordance with the present inventive concepts. FIG. 15B is a top perspective view of the base unit 200 with a bottom portion not illustrated (i.e. a portion of proximal base portion 210), in accordance with the present inventive concepts. Disposable probe assembly 100 and base unit 200 can be constructed and arranged as described in PCT Application No. PCT/US 16/28374 filed Apr. 20, 2016 entitled “ARTICULATED ROBOTIC PROBES, SYSTEMS AND METHODS INCORPORATING THE SAME, AND METHODS FOR PERFORMING SURGICAL PROCEDURES, the contents of which are incorporated herein by reference in their entirety.

Specifically, as shown in FIG. 15A, the proximal portion 105 of disposable assembly 100 includes one or more bobbins, bobbins 121 configured to mate with one or more pass-through capstans 221 of the base unit 200 (as shown below in FIG. 15B). Drive capstans of the base unit drive steering cables that are steerable by a cable control assembly, which advance or retract inner or outer links with respect to one another during manipulation of the probe assembly. The steering cables can be used to releasably tighten to lock or stiffen either or both of the plurality of inner links or the plurality of outer links of the articulating probe 150. Additionally, disposable probe assembly 100 can include a mating electrical connector port 131 that mates with a port 231 of base unit 200. Disposable probe assembly 100 can further include an inner probe connecter 175a and an outer probe connecter 185a. In some embodiments, inner and outer probe connectors 175a, 185a comprise projections having an interface surface configured to interface with corresponding interface surfaces 275a, 285a of inner and outer probe carriages 275, 285 (not shown) of base unit 200. The interface surfaces 275a, 285a are coupled to the inner and outer probe carriages 275, 285, which, when driven in the distal direction, apply a distal-oriented force to articulating probe 150.

In some embodiments, as shown in FIG. 15B, base unit 200 includes one or more aligmnent pins 241 configured to align with corresponding receiving holes 141 of disposable probe assembly 100. Alignment pins 241 and corresponding receiving holes 141 can be employed at one or more locations along the interface to ensure alignment and stability of at least a portion of disposable probe assembly 100 and base unit 200.

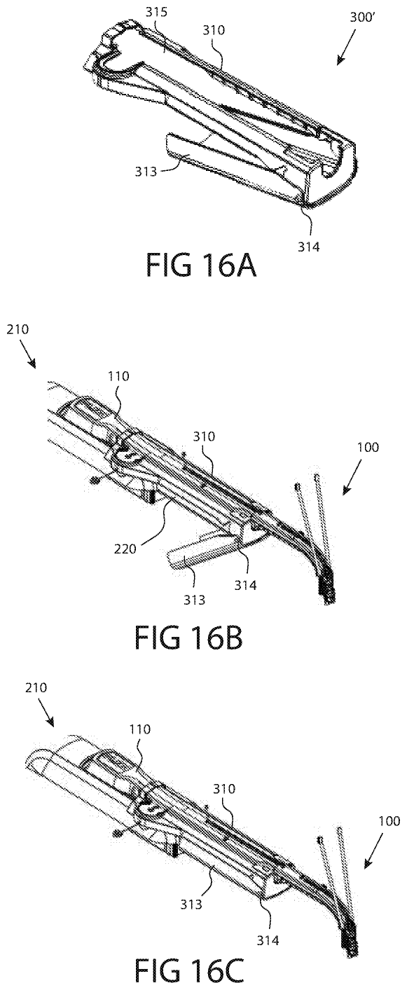

In FIGS. 16A-D, perspective views of an alternate embodiment of sterile frame 310 of FIGS. 4A and 4B is illustrated. Sterile barrier system 300′ includes frame 310 and a lower portion 313 operably attached to frame 310 via a hinge 314. Hinge 314 can be configured to rotatably attach lower portion 313 to frame 310.

In some embodiments, the distal end of robotic introducer assembly 50 (i.e. the distal portion of disposable probe assembly 100) is inserted through and exits opening 315 of sterile frame 310, and distal end 311 of sterile frame 310 is positioned proximate the distal end of base extension 220 (as described hereabove in refence to FIGS. 1-5). As shown in FIG. 16B, sterile frame 310 is positioned to align with and/or otherwise engage a mid-portion of disposable probe assembly 100 and/or proximal base portion 210. As shown in FIG. 16C, lower portion 313 is rotated, via hinge 314, to encapsulate a portion of robotic introducer assembly 50, and fixedly attach sterile barrier system 300′ to robotic introducer assembly 50. In some embodiments, frame 310 and/or lower portion 313 can comprise one or more attachment portions (e.g. clips), configured to connect lower portion 313 to frame 310 and/or a portion of robotic introducer system 50, for example, a snap-fit type connector.

In FIG. 17, an exploded perspective view of a carrier 60 for an articulating probe 110 of a disposable probe assembly is illustrated, in accordance with embodiments of the present inventive concepts. In FIGS. 17A-B, top and side views of an insert portion 65 of the carrier are illustrated, respectively. Carrier 60 can comprise packaging for the shipment of disposable probe assembly 100. In some embodiments, carrier 60 is constructed and arranged to receive disposable probe assembly 100. The proximal portion 105 of disposable probe assembly 100 can include one or more bobbins 121 (as discussed hereabove in reference to FIG. 15A). In some embodiments, carrier 60 includes an insert 65. As shown in FIGS. 17A and 17B, insert 65 can comprise one or more projections 66 configured to engage bobbins 121 of disposable probe assembly 100. Projections 66 of can be configured to prevent bobbins 121 from incidental rotation (e.g. during the shipment and handling of disposable probe assembly 100).

In some embodiments projections 66 can be configured to engage bobbins 121 such that projections 66 compress the internal springs of disposable probe assembly 100 by less than 40%, such as a compression of less than 30%, such that prolonged compression does not cause significant fatigue of the springs (e.g. in storage).

As described hereabove, sterile drapes 350, 450, 550, and 650 can comprise a material selected from the group consisting of: polyethylene; polyurethane; polycarbonate; and combinations thereof.

While the present inventive concepts have been particularly shown and described above with reference to exemplary embodiments thereof, it will be understood by those of ordinary skill in the art, that various changes in form and detail can be made without departing from the spirit and scope of the present inventive concepts described and defined by the following claims.

1. A system for performing a medical procedure within a sterile field, comprising:
a robotic introducer assembly including:
a base unit, the base unit including:
a proximal base portion; and
a base extension extending from the proximal base portion; and
a probe assembly operably and removably coupled to the base unit, the probe assembly including an articulating probe, wherein the base unit is constructed and arranged to control a movement of the articulating probe; and
the system further comprising:
a sterile barrier system positioned over at least a portion of the robotic introducer assembly, the sterile barrier system providing isolation between the sterile field and the at least a portion of the robotic introducer assembly, the sterile barrier system comprising:
a sterile frame having a first end coupled to a distal portion of the robotic introducer assembly and a second end coupled to a portion of the robotic introducer assembly proximal to a distal portion of the base unit; and
a sterile drape extending from the sterile frame and constructed and arranged for positioning about the base unit.

2.-117. (canceled)
